(19) United States
(12) Patent Application Publication
(10) Pub. No.: US 2006/0204535 A1
(43) Pub. Date: Sep. 14, 2006

(54) Cell-friendly cannula and needle

(76) Inventor: Johnnie M. Johnson, San Diego, CA (US)

Correspondence Address: FISH & RICHARDSON, PC, P.O. BOX 1022, MINNEAPOLIS, MN, 55440-1022, US

(21) Appl. No.: 11/363,568

(22) Filed: Feb. 27, 2006

Related U.S. Application Data
(60) Provisional application No. 60/655,823, filed on Feb. 25, 2005.

(51) Int. Cl. A61F 13/00

(52) U.S. Cl. 424/422; 427/2.1; 604/265

(57) ABSTRACT

An improved cannula and method for making the same are disclosed. The cannula includes a lumen having a polished inner surface, and a first hydrophilic coating on the polished inner surface of the lumen. The cannula can also include a polished outer surface, and a second hydrophilic coating on the polished outer surface. The first and second hydrophilic coatings can be one of a form of hydrogel or other hydrophilic polymer. The polishing is preferably done by one of several extrusion processes.

### CROSS REFERENCE TO RELATED APPLICATIONS

The present application claims priority under 35 U.S.C. §119 to U.S. Provisional Application Ser. No. 60/655,823, filed Feb. 25, 2005, entitled CELLFRIENDLY CANNULA AND NEEDLE, the disclosure of which is incorporated herein by reference

### BACKGROUND

A conventional cannula is a tube with a pointed, blunt or open tip at one end that is inserted through the skin or into a duct, vein, or cavity in order to harvest tissue and/or cells, drain away fluid or to administer drugs. A cannula can be flexible such as a hose, or rigid like a needle. Rigid cannulas have been utilized for many years for surgical procedures in which cells are collected by the cannula and removed from the patient. An example of such a procedure is liposuction, a cosmetic surgery in which fat cells are removed from under the skin by suction within the cannula.

Cannulas can also be used in procedures for harvesting a patient's autologous cells for reinjection or repositioning in the patient, such as medical procedures involving adult stem cells and cosmetic procedures involving adipose tissue. The use of autologous cells avoids many potential adverse effects when foreign substances are injected into a patient's body.

Cannulas are commonly attached to a syringe or machine that provides a vacuum to the tubular body of the cannula. The inner surface of the tubular body forms a lumen that can be extremely rough and jagged. The outer surface of the lumen might appear smooth to the touch or to the naked eye, but are also rough and jagged at the cellular level. These cannulas can yield a lower percentage of viable cells due to the trauma exerted upon cells that are forced into and through such a lumen. Further, during cell harvesting procedures the surrounding tissues are traumatized by the roughness of the outside surface of the cannula.

### SUMMARY

This document presents an apparatus and method that improves living tissue management. In particular, the disclosed apparatus and method minimizes trauma to the surrounding tissue and trauma to the cells removed for study or for harvesting and reinjection. Accordingly, improved cannulas and methods for making the same are presented. In one aspect, a cannula is formed by methods of extrusion and/or electropolishing that polish the inner surface of a lumen of the cannula and/or outer surface of the cannula to a high sheen to provide a slick and smooth surface. Both polished surfaces are then coated with a hydrophilic hydrogel.

In another aspect, a cannula includes a lumen having a polished inner surface, and a first hydrophilic coating on the polished inner surface. In another aspect, a cannula includes a polished outer surface, and a second hydrophilic coating on the polished outer surface. Each polished surface is polished by an extrusion process. Preferably, the hydraulic extrusion process includes hydraulically forcing an abrasive-laden polymer across each surface to be polished, thereby removing jagged and rough artifacts on those surfaces. In yet another aspect, each hydrophilic coating as described above further includes a polymer-based gel.

The improved cannulas allow for less traumatic removal of cells/tissue (hereafter referred to generally as “cells”) from a patient, and for harvesting greater amounts of viable cells for maintenance or reinjection back into the patient. Additionally, these cannulas can be connected to a vacuum source such as a syringe. In another aspect, the interior surface of the syringe is coated with a hydrophilic coating. The combination of improvements to both inner and outer surfaces provides for increased cell viability as they travel through the cannula and are stored in the syringe.

The details of one or more embodiments are set forth in the, accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIG. 1 is a cross sectional view of a cannula having a polished and coated outer surface, and a lumen with a polished and coated inner surface.

FIG. 2 is a perspective view of a cannula connected with a syringe as a vacuum source.

FIG. 3 is a flowchart of a method for making a cell-friendly cannula.

Like reference symbols in the various drawings indicate like elements.

### DETAILED DESCRIPTION

This document describes apparatuses and methods designed for the removal and harvesting of cells with significantly less trauma to the cells, the patient and surrounding tissue. As shown in FIG. 1, illustrating a cross-section of a cannula 100, the cannula 100 includes at least one lumen 102 having an inner surface 104. The lumen 102 preferably extends the entire length of the body of the cannula 100. The cannula 100 further includes an outer surface 106. The cannula 100 that forms the lumen 102 can be formed of any rigid or semi-rigid material, including, but not limited to, stainless steel, aluminum, titanium, nickel alloys, and any of a number of thermoplastics or composite fiber materials.

The inner surface 104 of the lumen 102 and/or outer surface 106 of the cannula 100 are polished to a microscopically smooth surface. According to embodiments, each polished surface is polished by an extrusion process or electropolishing. For example, an extrusion process used can include abrasive flow machining (AFM) and microflow AFM, processes in which a semisoft or viscous material (also known as “media”), such as an abrasive-laden polymer, is hydraulically forced over the surfaces that require polishing, such as the inner surface of the lumen and the exterior surface of the cannula. Preferably, the media has low viscosity, and preferably includes small abrasive particles. In some embodiments, the abrasive particles are maintained in the media at a substantially uniform distribution. Extrusion pressure is preferably controlled between 100 to 3,000 psi. Typically, a two-way flow AFM process is used, in which opposing cylinders extrude the media back and forth through the cannula. Alternatively, a one-way AFM flow process can be used for faster polishing and easier cleaning.

In accordance with embodiments, the cannula 100 further includes a first hydrophilic coating 108 on the polished inner surface 104 of the lumen 102. The cannula 100 in other embodiments includes a second hydrophilic coating 110 on the polished outer surface 106 of the cannula 100. The second hydrophilic coating 110 can also be provided on or around any openings into the lumen 102 of the body of the cannula 100.

Preferably, the hydrophilic coatings 108 and/or 110 include a polymer-based hydrogel for improved biocompatibility and lubricity for guiding and placement of the cannula 100 within tissue of a patient for cell-friendly and safe cell harvesting. The hydrogel coating preferably includes a combination of a polyvinylpyrrolidone with one of several isocyanate prepolymers. In some embodiments, a stable hydrophilic polymer coating includes a combination of a first polymer component, such as an organic solvent-soluble thermoplastic polyvinylbutyral (PVB), and a second polymer component, such as a hydrophilic poly (N-vinyl lactam), i.e. a water soluble polyvinylpyrrolidone. In other embodiments, other hydrogels can be used.

The hydrophilic coatings 108 and/or 110 reduce the tendency of platelets, proteins and encrustation to adhere to the inner and outer surfaces 104, 106 of the lumen 102 and cannula 100, respectively. Low-level heat is preferably used to cross-link and covalently bond the hydrogel coating 108, 110 to the inner and/or outer surfaces 104, 106 of the lumen 102 and cannula 100. The biocompatible, hydrophilic coatings 108, 110 swell instantaneously upon contact with water-containing fluids and tissue, and become highly lubricious and highly anti-thrombogenic. Further, the hydrophilic coatings 108 and 110 can be programmable for a controlled release of compounded or complex drugs and/or active agents for sustained, controlled local delivery to the cells when the cannula 100 is used. The hydrophilic coatings 108 and/or 110 could also be programmed with autologous (i.e. patient specific) seeding, including but not limited to platelet rich plasma (PRP), stem cells, and growth factors from blood cells, and endothelium from fat cells.

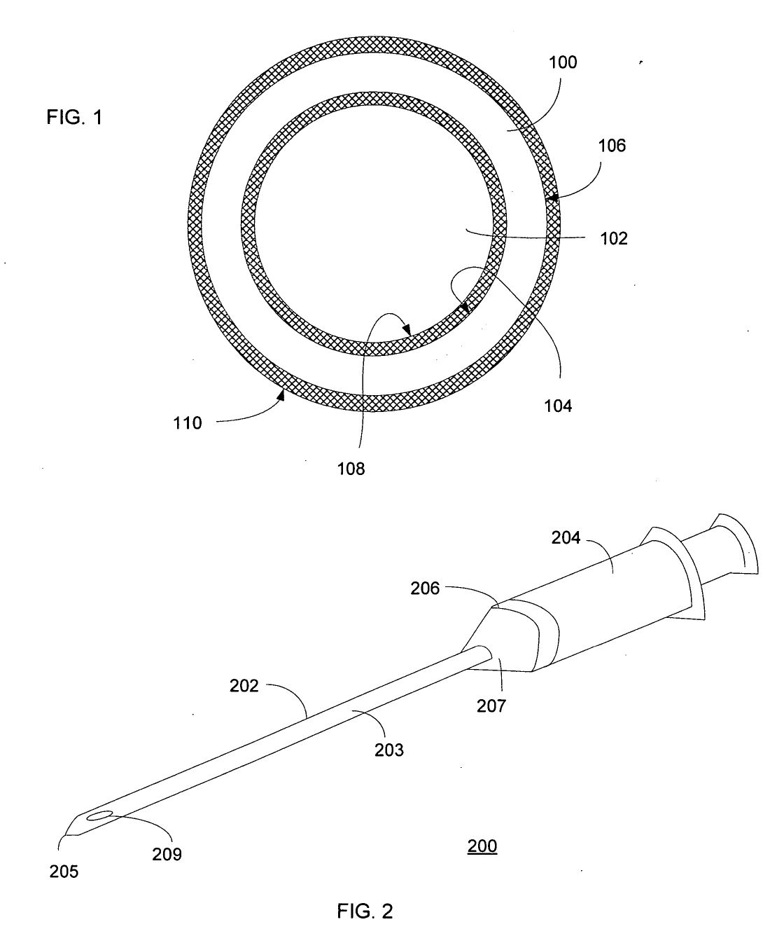

FIG. 2 shows a cell-friendly apparatus 200 that can be used for harvesting live cells without much trauma to the cells or surrounding tissue from which the cells are extracted. The apparatus 200 includes a cannula 202 connected with a vacuum and collection source 204, such as a syringe. The cannula 200 can be connected to the syringe via a leur lock 206 or other similar connecting mechanism.

The cannula 202 includes a tubular body 203 of various lengths having a distal end 205 with at least one opening or aperture 209 to a lumen extending the length of the tubular body 203. Fluid or cell tissue is harvested into the lumen via the opening or aperture 209, by physical manipulation and under force from the vacuum and collection source 204. The cannula 202 further includes a proximal end 207 that can be attached, for example, to the male or female end of the leur lock 206.

The outer surface of the cannula 202 and the inner surface of the lumen, as well as the surfaces defining and surrounding any opening or aperture 209, can be polished to a microscopically smooth surface, preferably according to one of a variety of extrusion processes as described above. Other polishing processes for deburring, radiusing, and surface finishing can be used. The polished surfaces are coated with a hydrophilic and cell-friendly coating, also as described above. Additionally, the interior surface of the vacuum and collection source 204 can be coated with the same or similar type of hydrophilic coating, such as a hydrogel.

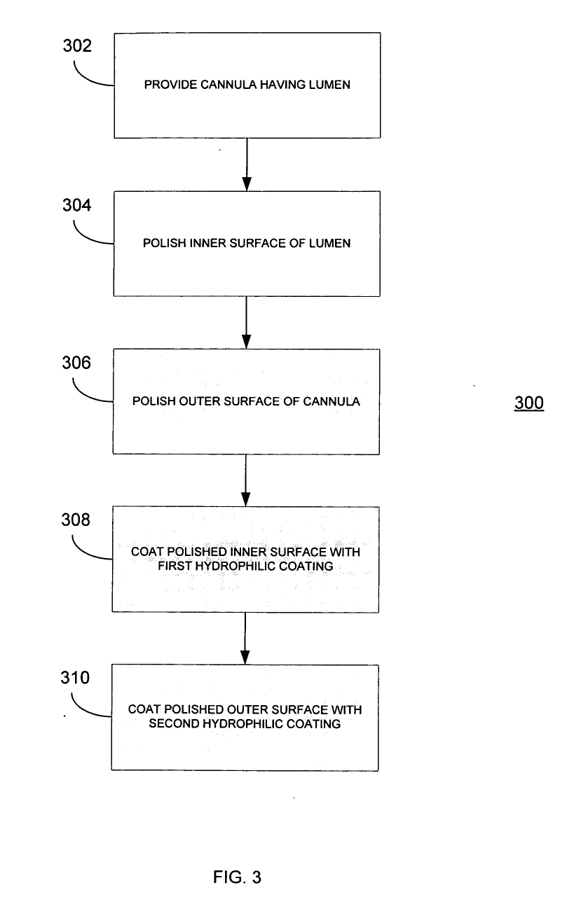

FIG. 3 is a flowchart of a method 300 for manufacturing an improved cannula as substantially described above. In accordance with preferred embodiments, method 300 includes providing a cannula having a lumen, at 302. The cannula can be formed of any rigid or semi-rigid material, including, but not limited to, stainless steel, aluminum, titanium, nickel alloys, and any of a number of thermoplastics or composite fiber materials. The cannula can be provided from any of a number of conventional manufacturing processes, to yield a tubular body of any of various lengths having a distal end with at least one opening or aperture to the lumen that extends the length of the tubular body, and a proximal end that can be attached, for example, to the female end of a leur lock for connection to a vacuum source such as a syringe or other machine.

At 304 the inner surface of the lumen, in whole or in part, is polished to remove rough or jagged edges or other artifacts, and to form the inner surface into a smooth surface. The polishing preferably includes an extrusion process such as an abrasive flow machining (AFM) or microflow AFM, process in which a semisoft or viscous media, such as an abrasive-laden polymer, is hydraulically forced over the inner surface that requires polishing, and/or electropolishing method. At 306, the outer surface of the cannula, in whole or in part, is similarly polished. The polishing steps of 304 and 306 can occur simultaneously within a single polishing process, or separately in either order or according to different polishing processes.

At 308, the polished inner surface of the lumen is coated, in whole or in part, with a first hydrophilic coating. The hydrophilic coating can be formed of any substance used for protecting the viability of live cells/tissue. The hydrophilic coating preferably includes a polymer-based hydrogel. The hydrogel coating can include, for example, a combination of a polyvinylpyrrolidone with one of several isocyanate prepolymers. In some embodiments, a stable hydrophilic polymer coating includes a combination of a first polymer component, such as an organic solvent-soluble thermoplastic polyvinylbutyral (PVB), and a second polymer component, such as a hydrophilic poly (N-vinyl lactam), such as a water soluble polyvinylpyrrolidone.

At 310, the polished outer surface of the cannula is coated, in whole or in part, with a second hydrophilic coating. The second hydrophilic coating can be the same hydrogel as the first hydrophilic coating, or may be different from the first hydrophilic coating. The first and/or second hydrophilic coatings can be programmable, i.e. laden with compounds or complex drugs and/or active agents of any desired quantity or level, for sustained, controlled local delivery to cells. The compounds and/or complexes can be added to the hydrophilic coating before, during, or after application to any surface, in the same or separate steps. The steps 308 and 310 can be executed simultaneously, or one after the other in either order.

Accordingly, a cannula is produced having slick and hydrophilic properties, that exert less trauma to harvested cells or surrounding tissue during cell harvesting operations than conventional cannulas. Although a few embodiments have been described in detail above, other modifications are possible. The flows depicted in FIG. 3 do not require the particular order shown, or sequential order, to achieve desirable results. Other embodiments may be within the scope of the following claims.

1. A cannula comprising:
a lumen having a polished inner surface; and
a first hydrophilic coating on the polished inner surface of the lumen.

2. A cannula in accordance with claim 1, further comprising a polished outer surface.

3. A cannula in accordance with claim 2, further comprising a second hydrophilic coating on the polished outer surface of the cannula.

4. A cannula in accordance with claim 1, wherein the polished inner surface is polished by an extrusion process.

5. A cannula in accordance with claim 2, wherein the polished outer surface is polished by an extrusion process.

6. A cannula in accordance with claim 1, wherein the first hydrophilic coating includes a polymer-based gel.

7. A cannula in accordance with claim 3, wherein the second hydrophilic coating includes a polymer-based gel.

8. A cannula in accordance with claim 6 or 7, wherein the polymer-based gel includes a combination of a polyvinylpyrrolidone with a isocyanate prepolymer.

9. A cannula in accordance with claim 8, wherein the combination of a polyvinylpyrrolidone with a isocyanate prepolymer includes a thermoplastic polyvinylbutyral (PVB) combined with a hydrophilic poly (N-vinyl lactam).

10. A cannula in accordance with claim 1, wherein the cannula further comprises a distal end having at least one opening, and a proximal end having an interface to a vacuum source.

11. A cannula in accordance with claim 1, wherein the first hydrophilic coating can be programmed for a controlled release of a complex agent.

12. A cannula in accordance with claim 1, wherein the first hydrophilic coating can be programmed with an autologous seeding.

13. An apparatus for harvesting live cells, comprising:
a cannula having a lumen that includes a polished inner surface and a first hydrophilic coating on the polished inner surface; and
a syringe connected to the cannula and having an inner surface and a second hydrophilic coating on the inner surface.

14. An apparatus in accordance with claim 13, wherein the first hydrophilic coating includes a polymer-based gel.

15. An apparatus in accordance with claim 13, wherein the second hydrophilic coating includes a polymer-based gel.

16. An apparatus in accordance with claim 14 or 15, wherein the polymer-based gel includes a combination of a polyvinylpyrrolidone with a isocyanate prepolymer.

17. An apparatus in accordance with claim 16, wherein the combination of a polyvinylpyrrolidone with a isocyanate prepolymer includes a thermoplastic polyvinylbutyral (PVB) combined with a hydrophilic poly (N-vinyl lactam).

18. An apparatus in accordance with claim 13, wherein the cannula further comprises a distal end having at least one opening, and a proximal end having a female leur lock interface for connection to the syringe.

19. A method of treating a cannula comprising:
polishing an inner surface of a lumen of the cannula; and
coating the polished inner surface of the lumen with a first hydrophilic coating.

20. A method in accordance with claim 19, wherein polishing the inner surface of the lumen of the cannula comprises extruding the inner surface with an abrasive-laden polymer media.

21. A method in accordance with claim 19, further comprising:
polishing an outer surface of the cannula; and
coating the polished outer surface of the cannula with a second hydrophilic coating.

22. A method in accordance with claim 21, wherein polishing the outer surface of the cannula comprises extruding the outer surface with an abrasive-laden polymer media.
